(19) United States
(12) Patent Application Publication
(10) Pub. No.: US 2022/0265522 A1
(43) Pub. Date: Aug. 25, 2022

(54) Lotion Pellet and Method of Manufacture

(71) Applicants: Marvin Hartley, West Point, UT (US); Kathleen Hartley, West Point, UT (US)

(72) Inventors: Marvin Hartley, West Point, UT (US); Kathleen Hartley, West Point, UT (US)

(21) Appl. No.: 17/182,261

(22) Filed: Feb. 23, 2021

(51) Int. Cl. A61K 8/02; A61K 8/92; A61K 8/9794; A61Q 19/00

(52) U.S. Cl. CPC A61K 8/0216; A61K 8/922; A61K 8/9794; A61Q 19/007; A61K 2800/805; A61K 2800/59

(57) ABSTRACT

A method of manufacturing a lotion pellet, and a lotion pellet, is disclosed. The method comprises the steps of mixing a combination of coconut oil, cocoa butter, shea butter and beeswax, over heat, for a period of time to form an integrated mixture of the combination, providing a mold having a mold cavity, poring an amount of the integrated mixture into the mold cavity, allowing the poured amount of the integrated mixture to settle in the mold cavity for a period of time, chilling the integrated mixture in the mold for a period of time, to form the lotion pellet, and removing the lotion pellet from the mold cavity.

### FIELD OF THE INVENTION

The present invention relates to a hand lotion formed as semi-solid body, having a firm consistency, and a method of manufacturing thereof.

### BACKGROUND

People often have skin conditions, such as eczema or simply dry skin, which can be uncomfortable or even painful, particularly during certain seasons. Some products, such as prescription medications, and even some sold over the counter, can be expensive and even themselves irritating. Often too, the products can be difficult and/or inconvenient to carry and/or apply.

The present invention is provided to solve these and other problems.

### DESCRIPTION OF THE FIGURES

For a more complete understanding of the disclosure, reference should be made to the following detailed description.

### DESCRIPTION OF THE PREFERRED EMBODIMENT

While this invention is susceptible of embodiment in many different forms, there will be described herein in detail, a specific embodiment thereof with the understanding that the present disclosure is to be considered an exemplification of the principles of the invention and is not intended to limit the invention to the specific embodiment illustrated.

In accordance with the present invention, a method of manufacturing a lotion pellet, and the lotion pellet itself is herein described.

The method may comprise the step of placing a combination comprising coconut oil, cocoa butter, shea butter and beeswax, into a container, such as a double boiler. The preferred ratio of coconut oil (in fluid ounces, or fl oz) to cocoa butter (fl oz) to shea butter (fl oz) to beeswax (in ounces, or oz) is preferably of the order of 2:1:1:2. For example, one batch of product may be made by combining 32 fl oz of coconut oil, 16 fl oz of cocoa butter, 16 fl oz of shea butter, and 32 oz of beeswax, each of which are common, off the shelf items. The components are preferably all natural, absent chemical additives.

While the above relative amounts are, as indicated, preferred, satisfactory results may be obtained utilizing: coconut oil in the range of 24-38 fl oz; cocoa butter in the range of 11-20 fl oz; shea butter in the range of 12-20 fl oz; and beeswax in the range of 24-36 oz. Too much coconut oil may result in a product too slimy or slick. Too much cocoa butter may result in a product which smells too much like chocolate. Too much shea butter may result in a product too sticky. Too much beeswax may result in a product too hard.

Should a scented lotion pellet be desired, 1-4 fl oz (relative to the above component amounts) of a skin safe, naturally refined essential oil may be added to the combination, depending upon a desired fragrance level. The essential oils may include mahogany teakwood, black raspberry vanilla and white peach/hibiscus.

The combination may then be heated in the double boiler to a temperature in the range of 180° F.-225° F., preferably to a temperature of the order of 210° F. Overheating the mixture may result in an undesirable burning of the mixture.

The combination may then be mixed, as it is heated, for a period of time sufficient to form an integrated mixture. This period of time is preferably about five minutes, as any longer may tend to boil of the essential oils, if included.

A mold may then be provided. The mold may have one or more, generally hemispherical, mold cavities. The mold cavities may be preferably about the size of a pea. One such mold may be the JeVenis 468 Cavity Chocolate Drops Mold, available on Amazon.

In a next step, an amount of the integrated mixture may be poured into the one or more mold cavities, and the poured amount of the integrated mixture may be allowed to settle in the one or more mold cavities for a period of time of approximately five minutes.

The integrated mixture in the one or more mold cavities may then be chilled at a temperature of preferably −10° F. (+/−20° F.) for a period of time of to permit the integrated mixture to solidify, which may be approximately two hours, to form a lotion pellet having a firm consistency.

Once formed to the proper consistency, the one or more pellets may be removed from their respective one or more mold cavities.

In use one may place one-half to an entire one of the lotion pellets into the palm of one's hand and smash the pellet with the thump of the opposite hand. Heat from one's body will soften lotion pellet. One may rub hands up and down, and to one's arms if desired. One may wait two-three minutes, and lotion from the lotion pellet will be absorbed into one's skin. The lotion pellet may be used on legs, feet and other parts of the body.

The lotion pellets are for external use only.

It is to be understood that this disclosure is not intended to limit the invention to any particular form described, but to the contrary, the invention is intended to include all modifications, alternatives and equivalents falling within the spirit and scope of the invention.

We claim:

1. A method of manufacturing a lotion pellet comprising the steps of:
mixing a combination of coconut oil, cocoa butter, shea butter and beeswax, over heat, for a period of time to form an integrated mixture of the combination;
providing a mold having a mold cavity;
poring an amount of the integrated mixture into the mold cavity;
allowing the poured amount of the integrated mixture to settle in the mold cavity for a period of time;
chilling the integrated mixture in the mold for a period of time, to form the lotion pellet; and
removing the lotion pellet from the mold cavity.

2. The method of claim 1, wherein the ratio of coconut oil to cocoa butter to shea butter to beeswax is of the order of 2 fl oz:1 fl oz:2:fl oz:1 oz.

3. The method of claim 1, wherein the relative ratio of the components is coconut oil in the range of 24-38 fl oz; cocoa butter in the range of 11-20 fl oz; shea butter in the range of 12-20 fl oz; and beeswax in the range of 24-36 oz.

4. The method of claim 1, wherein the combination is heated to a temperature in the range of 180° F.-225° F.

5. The method of claim 4, wherein the combination is heated to a temperature of the order of 210° F.

6. The method of claim 4, wherein the combination is heated for a period of time of approximately five minutes.

7. The method of claim 1, wherein the integrated mixture is chilled at a temperature of approximately −10° F.

8. The method of claim 1, wherein the integrated mixture is chilled to permit solidification of the integrated mixture

9. The method of claim 1, wherein the integrated mixture is permitted to settle for at least a minimum time of five minutes.

10. The method of claim 1, wherein the combination includes an essential oil.

11. A lotion pellet formed by the method of claim 1.

11. A method of manufacturing a lotion pellet comprising the steps of:
placing a combination comprising coconut oil, cocoa butter, shea butter and beeswax, into a container;
heating the combination to a temperature of the order of 210° F.;
mixing the combination as it heats to form an integrated mixture;
providing a mold having a mold cavity;
poring an amount of the integrated mixture into the mold cavity;
allowing the poured amount of the integrated mixture to settle in the mold cavity for a period of time;
chilling the integrated mixture in the mold for a period of time sufficient to permit the integrated mixture to substantially solidify, to form a lotion pellet having a firm consistency; and
removing the lotion pellet from the mold cavity.

12. The method of claim 11, wherein the relative ratio of the components is coconut oil in the range of 24-38 fl oz; cocoa butter in the range of 11-20 fl oz; shea butter in the range of 12-20 fl oz; and beeswax in the range of 24-36 oz.

13. The method of claim 11, wherein the integrated mixture is allowed to settle for approximately five minutes.

14. The method of claim 11, wherein the integrated mixture is chilled at a temperature of the order of −10° F.

15. The method of claim 11, wherein the integrated mixture is chilled for at a temperature of the order of −10° F. for a period of time of the of the order of twelve hours.

16. The method of claim 11, wherein the combination includes an essential oil.

17. A lotion pellet formed by the method of claim 11.

18. A method of manufacturing a lotion pellet comprising the steps of:
placing a combination comprising coconut oil, cocoa butter, shea butter and beeswax, into a container, wherein the ratio of coconut oil to cocoa butter to shea butter to beeswax is of the order of 2 fl oz:1 fl oz:1 fl oz:2 oz.;
heating the combination to a temperature of the order of 210° F.;
mixing the combination as it heats for a period of time to form an integrated mixture;
providing a mold having a mold cavity;
poring an amount of the integrated mixture into the mold cavity;
allowing the poured amount of the integrated mixture to settle in the mold cavity for a minimum period of time of approximately five minutes;
chilling the integrated mixture in the mold at a temperature of less than −10° F. for a period of time to substantially solidify, to form a lotion pellet having a firm consistency; and
removing the lotion pellet from the mold cavity.

19. The method of claim 18, wherein the combination is heated in a double boiler.

20. The method of claim 18, wherein the combination includes an essential oil.

21. A lotion pellet formed by the method of claim 18.
